(19) United States
(12) Patent Application Publication
(10) Pub. No.: US 2017/0000956 A1
(43) Pub. Date: Jan. 5, 2017

(54) SAFETY SYRINGE ASSEMBLY

(71) Applicants: Ching Hsiang Wei, Taichung City (TW); Jun He Technology Co., Ltd, Changhua County (TW)

(72) Inventors: Ching Hsiang Wei, Taichung City (TW); Cheng-Hua Hsieh, Changhua County (TW)

(21) Appl. No.: 14/791,453

(22) Filed: Jul. 5, 2015

(51) Int. Cl. A61M 5/32

(52) U.S. Cl. CPC A61M 5/3271; A61M 5/3245; A61M 5/3257

(57) ABSTRACT

A safety syringe assembly includes a syringe and a concealing apparatus. The syringe includes a cylinder, two wings formed on two opposite sides of the cylinder, a needle connected to a front end of the cylinder, and a tube connected to a rear end of the cylinder. The concealing apparatus includes a guiding element and two covers. The guiding element is located around the tube. The two covers are engaged with each other and movable along the guiding element between an original position for exposing the guiding element and a concealing position for concealing the needle.

### BACKGROUND OF INVENTION

1. Field of Invention

The present invention relates to a syringe assembly and, more particularly, to a safety syringe assembly including a protective sleeve for completely covering a needle after use.

2. Related Prior Art

To protect a medical worker from a needle, it is necessary to provide a protective mechanism for covering the needle after a syringe is used. Winged needles such as venoclysis needles and intravenous indwelling needles are often used with protective mechanisms. Taiwanese Patent M257833 discloses a sleeve that includes a long slit extending to a front end. The wing or wings of a venoclysis needle can be folded and inserted in the sleeve via the slit. Taiwanese Patent M489646 discloses a box provided with a lid. The box is used to contain a venoclysis needle and the lid is used to close the box. The box is bulky. Moreover, the box interferes with the handling of the venoclysis needle because the box is provided on a transfusion tube connected to the venoclysis needle. Taiwanese Patent M496494 discloses a cover movably connected to a root of a needle. The cover is in an exposing position to expose the needle. The cover can be moved to a concealing position to conceal the entire needle. However, in the exposing position, the cover affects the visibility of the needle and hence interferes with the handling of the needle.

The present invention is therefore intended to obviate or at least alleviate the problems encountered in prior art.

### SUMMARY OF INVENTION

It is the primary objective of the present invention to provide a safety syringe assembly.

To achieve the foregoing objective, the safety syringe assembly includes a syringe and a concealing apparatus. The syringe includes a cylinder, two wings formed on two opposite sides of the cylinder, a needle connected to a front end of the cylinder, and a tube connected to a rear end of the cylinder. The concealing apparatus includes a guiding element and two covers. The guiding element is located around the tube. The covers are engaged with each other and movable along the guiding element between an original position around the guiding element and a concealing position for concealing the needle.

The concealing apparatus can advantageously be used for a venoclysis needle or an intravenous indwelling needle.

The covers do not interfere with the handling of the syringe when they are in the original position.

Moreover, after the syringe is used, the covers are moved to the concealing position to conceal the entire needle to prevent the needle from hurting anybody.

Furthermore, the covers cannot be disengaged from each other to ensure that the needle is concealed by the covers.

In another aspect, the guiding element is a rectangular cylinder to render the connection of the covers thereto firm.

In addition, the rectangular cylindrical configuration of the guiding element reduces the size of the concealing apparatus but increases the strength of the concealing apparatus.

Moreover, the movement of the covers can be done with a single hand.

In another aspect, each of the covers includes a pattern for anti-skid and direction-indicating purposes.

Other objectives, advantages and features of the present invention will be apparent from the following description referring to the attached drawings.

### BRIEF DESCRIPTION OF DRAWINGS

The present invention will be described via detailed illustration of the preferred embodiment referring to the drawings wherein:

FIG. 1 is an exploded view of a safety syringe assembly according to the preferred embodiment of the present invention;

FIG. 2 is another exploded view of the safety syringe assembly shown in FIG. 1;

FIG. 3 is a perspective view of the safety syringe assembly shown in FIG. 1;

FIG. 4 is an enlarged partial side view of the safety syringe assembly shown in FIG. 3;

FIG. 5 is an enlarged partial cross-sectional view of the safety syringe assembly shown in FIG. 4;

FIG. 6 is a perspective view of the safety syringe assembly in another position than the position shown in FIG. 3;

FIG. 7 is an enlarged partial side view of the safety syringe assembly shown in FIG. 6;

FIG. 8 is an enlarged partial cross-sectional view of the safety syringe assembly shown in FIG. 7; and

FIG. 9 is an enlarged partial side view of the safety syringe assembly in another position than shown in FIG. 7.

### DETAILED DESCRIPTION OF PREFERRED EMBODIMENT

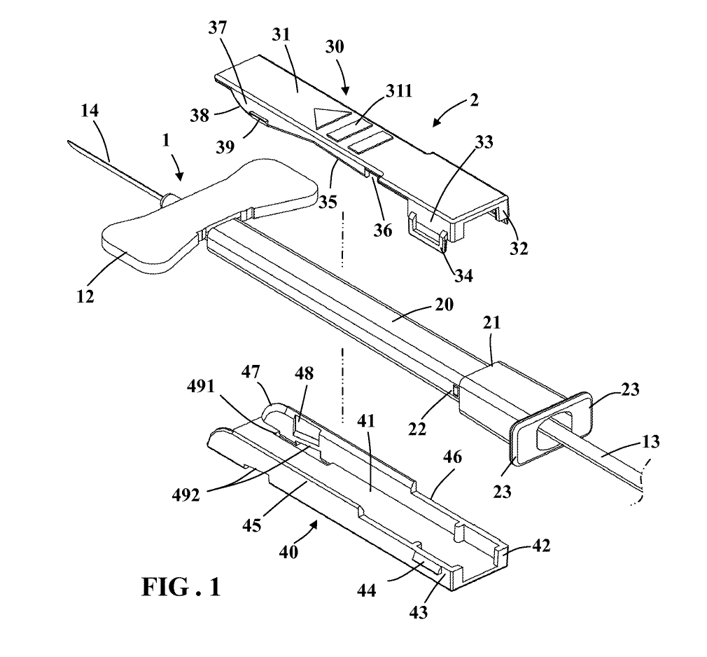

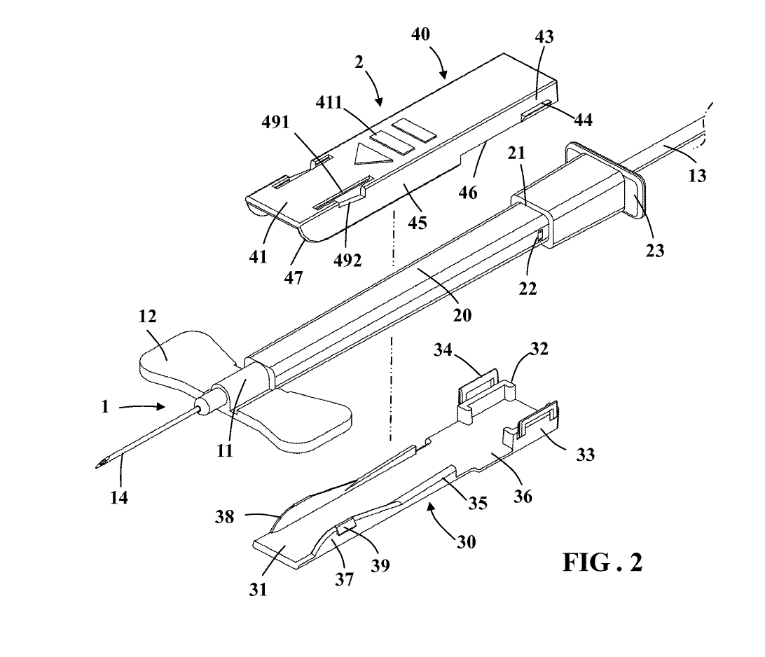

Referring to FIGS. 1 and 2, a safety syringe assembly includes a syringe 1 and a concealing apparatus 2 according to the preferred embodiment of the present invention. The syringe 1 is a winged syringe such as a venoclysis syringe and an intravenous indwelling syringe. The syringe 1 includes a cylinder 11, two wings 12, a tube 13 and a needle 14. The wings 12 are formed on two opposite sides of the cylinder 11. The tube 13 is connected to a rear end of the cylinder 11. The needle 14 is connected to a front end of the cylinder 11. Medicine in liquid form is transferred into the cylinder 11 from the tube 13 and then into a vein from the needle 14. The concealing apparatus 2 is connected to the syringe 1 and includes a guiding element 20 and two covers 30 and 40.

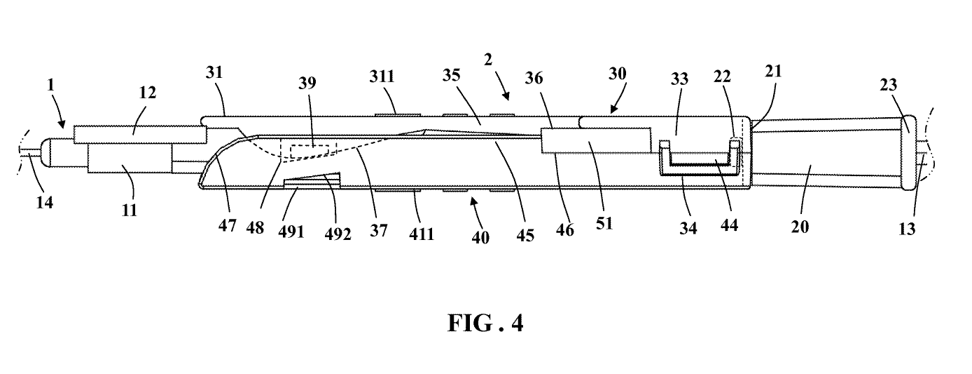

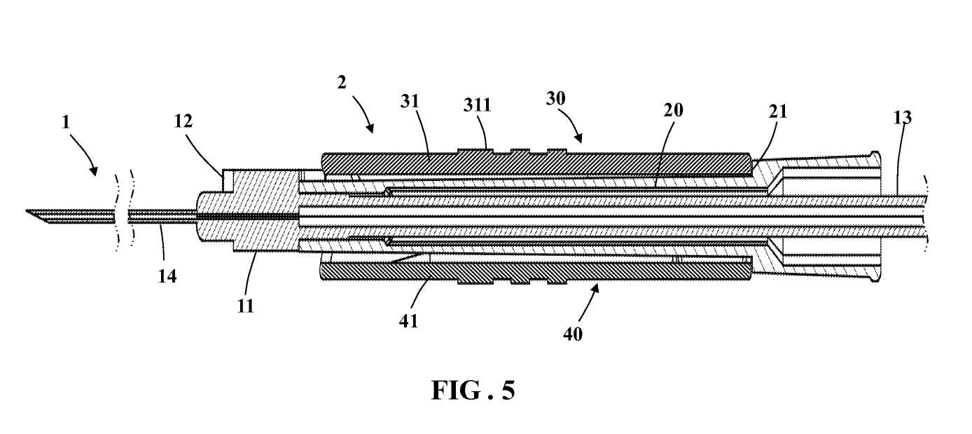

Referring to FIGS. 1 to 5, the guiding element 20 is a rectangular tubular element provided around a section of the tube 13. The guiding element 20 includes a stop 21 formed between two sections of difference dimensions. Two bosses 22 are formed on two opposite sides of the first section of the guiding element 20. The fins 23 are formed on two opposite sides of the second section of the guiding element 20.

The first cover 30 includes a plate 31 that extends horizontally. For anti-skid and direction-indicating purposes, a pattern 311 is formed on an external face of the plate 31. Two vertical rear end strips 32 and two vertical rear lateral strips 33 are formed on a rear section of an internal face of the plate 31. Each of the rear end strips 32 is connected to a corresponding one of the rear lateral strips 33. A buckle 34 is formed on each of the rear lateral strips 33. Two vertical middle lateral strips 35 are formed on a middle section of the internal face of the plate 31. There is a gap 36 between each of the middle lateral strips 35 and a corresponding one of the rear lateral strips 33. Two hooking strips 37 are formed on a front section of the plate 31. Each of the hooking strips 37 is connected to a corresponding one of the middle lateral strips 35. Each of the hooking strips 37 includes an arched convex guiding edge 38. A barb 39 is formed on an external side of each of the hooking strips 37.

The second cover 40 includes a plate 41 that extends horizontally. For anti-skid and direction-indicating purposes, a pattern 411 is formed on an external face of the plate 41. Two rear end strips 42 and two rear lateral strips 43 are formed on a rear section of an internal face of the plate 41. Each of the rear end strips 42 is connected to a corresponding one of the rear lateral strips 43. A barb 44 is formed on an external side of each of the rear lateral strips 43. Two vertical buckling strips 45 are formed on a front portion of the internal face of the strip 41. There is a gap 46 between each of the buckling strips 45 and a corresponding one of the rear lateral strips 43. Each of the buckling strips 45 includes an arched convex guiding edge 47, a recess 48 made in an internal side, and an aperture 492 in communication with a corresponding one of two slots 491 made in the plate 41.

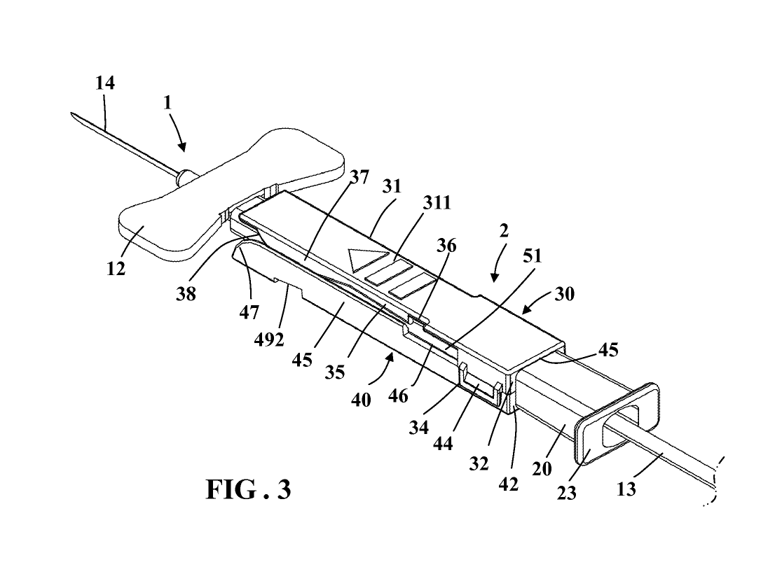

Referring to FIGS. 3 to 5, the buckles 34 of the first cover 30 are engaged with the barbs 44 of the second cover 40 to firmly connect the first cover 30 to the second cover 40. The combination of the first cover 30 with the second cover 40 is located around the guiding element 20. Now, the covers 30 and 40 are in an original position between the stop 21 of the guiding element 20 and the cylinder 11 of the syringe 1. When the covers 30 and 40 are in the original position, the needle 14 of the syringe 1 is exposed for use. The rear end strips 32 of the first cover 30 and the rear end strips 42 of the second cover 40 are located between the stop 21 of the guiding element 20 and the bosses 22 of the same to keep the covers 30 and 40 in the original position. The covers 30 and 40 will not be moved away from the original position without being subjected to an adequate force. Each of the gaps 36 of the first cover 30 and a corresponding one of the gaps 46 of the second cover 40 together form a slot 51. Each of the buckling strips 45 of the second cover 40 is located in the vicinity of a corresponding one of the hooking strips 37 of the first cover 30. The hooking strips 37 of the first cover 30 are located between the buckling strips 45 of the second cover 40. The barbs 39 of the hooking strips 37 of the first cover 30 are inserted in the recesses 48 of the buckling strips 45 of the second cover 40.

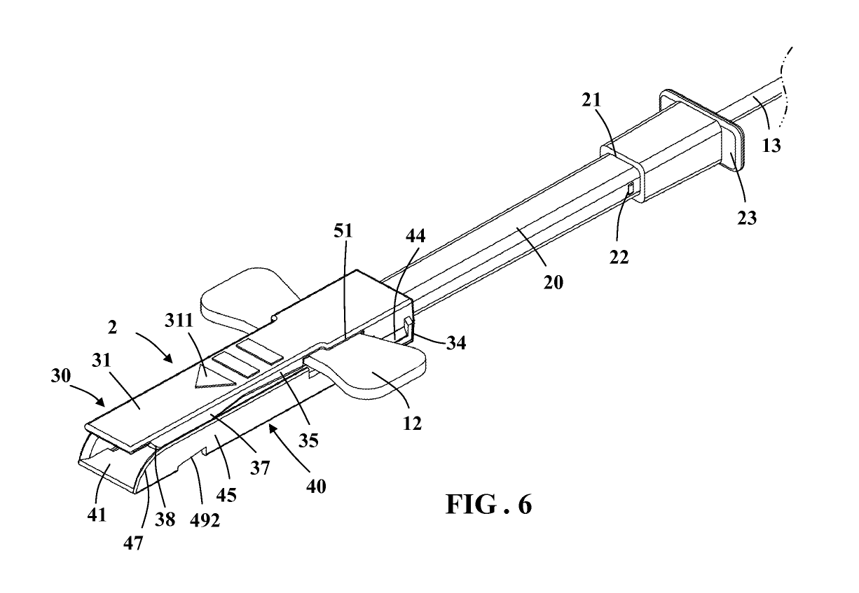

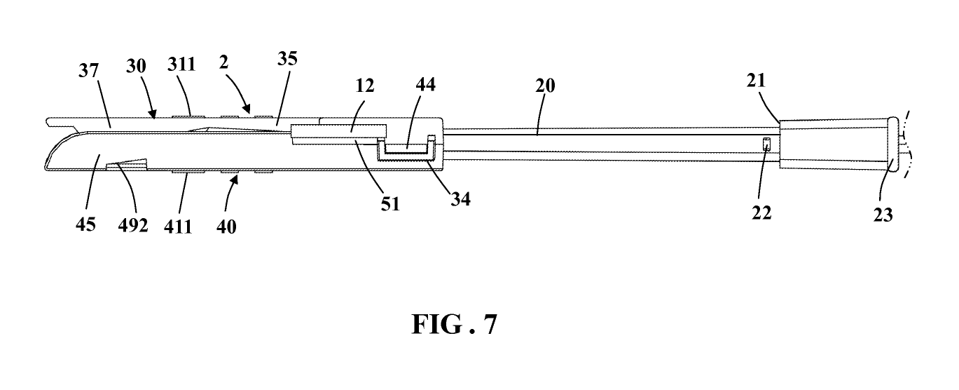

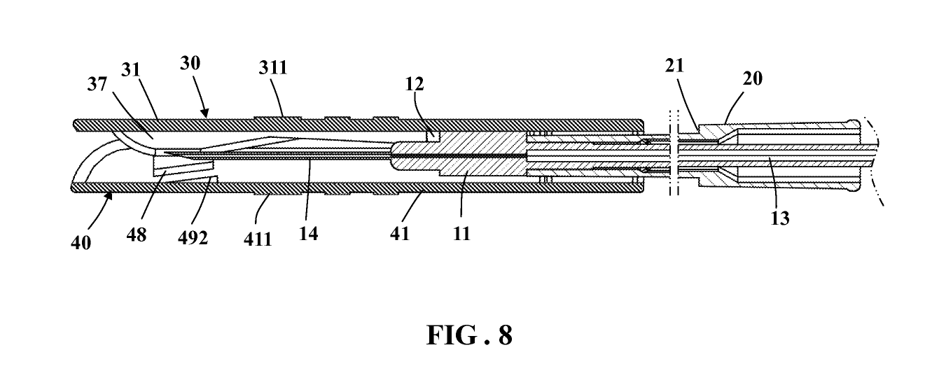

Referring to FIGS. 6 to 8, after use, the needle 14 is concealed by the concealing apparatus 2 to prevent it from hurting any person. To this end, the guiding element 20 can be pulled rearward or the covers 30 and 40 are moved forward. Anyway, the covers 30 and 40 are moved forward relative to the guiding element 20. A user can achieve this operation with a singled hand. The user can hold the covers 30 and 40 with the thumb and the index finger, contact a rear section of the guiding element 20 with the palm, and push the fins 23 of the guiding element 20 with the middle finger or the ring finger. An adequate force must be exerted when the movement just begins because the bosses 22 of the guiding element 20 are in contact with the rear end strips 32 and 42 of the covers 30 and 40 to resist the movement. The adequate force moves the rear end strips 32 and 42 of the covers 30 and 40 past or over the bosses 22 of the guiding element 20. Then, the covers 30 and 40 can be smoothly moved along the guiding element 20. Finally, a front section of the concealing apparatus 2 is moved past the wings 12 of the syringe 1 because of the guiding edges 38 and 47. When the hooking strips 37 and middle lateral strips 35 of the first cover 30 and the buckling strips 45 of the second cover 40 are moved past the wings 12, a front section of the first cover 30 is moved away from a front section of the second cover 40 because of the thickness of the wings 12. Then, the wings 12 are inserted in the slots 51 made by the gaps 36 and 46 of the covers 30 and 40. Now, the covers 30 and 40 are in a concealing position where the covers 30 and 40 completely conceal the needle 14. The wings 12 are kept in the slots 51 since a front edge of each of the wings 12 is confined by a corresponding one of the rear lateral strips 33 and a corresponding one of the rear lateral strips 43 and a rear edge of each of the wings 12 is confined by a corresponding one of the middle lateral strips 35 and a corresponding one of the buckling strips 45. Hence, the needle 14 is kept in the covers 30 and 40, i.e., the needle 14 cannot be exposed again.

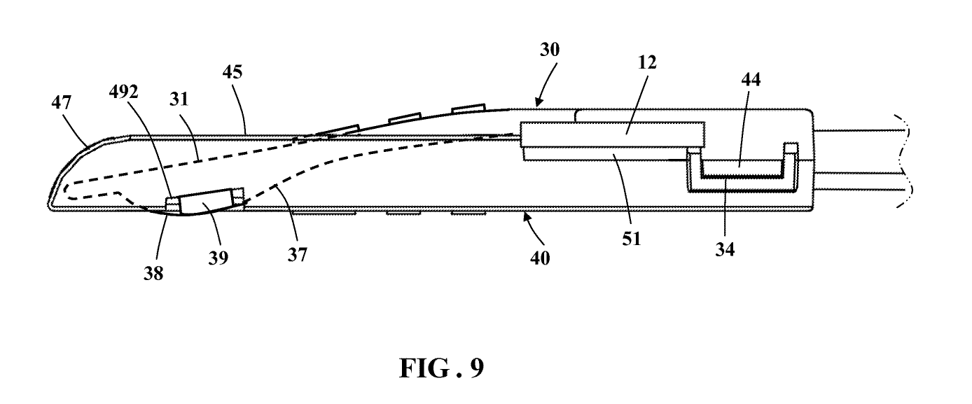

Referring to FIG. 9, the front section of the first cover 30 can be engaged with the front section of the second cover 40 to avoid movement of the covers 30 and 40 from each other. The user uses the thumb and the index finger to press the front sections of the covers 30 and 40 to move the barbs 39 of the hooking strips 37 of the first cover 30 out of the recesses 48 of the buckling strips 48 of the second cover 40. Each of the barbs 39 includes a slope to facilitate the movement of the barb 39 past or over a wall formed next to a corresponding one of the recesses 48, press the hooking strips 37 of the first cover 30, and enable the corresponding hooking strip 37 together with the barb 39 to move past the internal side of the corresponding buckling strip 45 of the second cover 40. Then, the barbs 39 enter the apertures 492 while the guiding edges 38 of the hooking strips 37 of the first cover 30 are inserted in the slots 491 of the second cover 40. Thus, the front sections of the covers 30 and 40 are engaged with each other. Now, the front section of the plate 31 of the first cover 30 extends in an arched manner. The engagement of the front sections of the covers 30 and 40 with each other is unbreakable.

The present invention has been described via the detailed illustration of the preferred embodiment. Those skilled in the art can derive variations from the preferred embodiment without departing from the scope of the present invention. Therefore, the preferred embodiment shall not limit the scope of the present invention defined in the claims.

1. A safety syringe assembly comprising:
a syringe (1) comprising:
a cylinder (11);
two wings (12) extending from two opposite sides of the cylinder (11);
a needle (14) connected to an end of the cylinder (11); and
a tube (13) connected to another end of the cylinder (11); and
a concealing apparatus (2) comprising:
a guiding element (20) located around the tube (13) and formed with a stop (21);
a first cover (30) comprising:
a plate (31);
two rear lateral strips (33) formed on the plate (31);
two buckles (34) each formed on a corresponding one of the rear lateral strips (33);
two hooking strips (37) formed on the plate (31) and each made with a barb (39); and
two gaps (36) each made between a corresponding one of the hooking strips (37) and a corresponding one of the rear lateral strips (33); and
a second cover (40) comprising:
a plate (41);
two rear lateral strips (43) formed on the plate (41) of the second cover (40) and each made with a barb (44) engaged with a corresponding one of the buckles (34) of the first cover (30);
two buckling strips (45) formed on the plate (41) of the second cover (40) and each engaged with the barb (39) of a corresponding one of the hooking strips (37) of the first cover (30); and
two gaps (46) each made between a corresponding one of the buckling strips (45) and a corresponding one of the rear lateral strips (43) of the second cover (40), wherein each of the gaps (46) of the second cover (40) and a corresponding one of the gaps (36) of the first cover (30) together become a slot (51) for movably receiving a corresponding one of the wings (12) of the syringe (1) when the first cover (30) and the second cover (40) are joined together;
wherein the first cover (30) and the second cover (40) are movable along the guiding element (20) between an original position around the guiding element (20) and a concealing position for completely concealing the needle (14).

2. The safety syringe assembly according to claim 1, wherein the guiding element (20) is a tubular element.

3. The safety syringe assembly according to claim 1, wherein the guiding element (20) is formed with two bosses (22), and the first cover (30) comprises two rear end strips (32) each located between the stop (21) and a corresponding one of the bosses (22) when the first cover (30) and the second cover (40) are in the original position.

4. The safety syringe assembly according to claim 1, wherein the guiding element (20) is formed with two fins (23) for facilitating handling thereof.

5. The safety syringe assembly according to claim 1, wherein the first cover (30) comprises a pattern (311) formed on the plate (31) thereof, wherein the pattern (311) is selected from the group consisting of an anti-skid pattern and a direction-indicating pattern.

6. The safety syringe assembly according to claim 1, wherein the second cover (40) comprises a pattern (411) formed on the plate (41) thereof, wherein the pattern (411) is selected from the group consisting of an anti-skid pattern and a direction-indicating pattern.

7. The safety syringe assembly according to claim 1, wherein each of the buckling strips (45) comprises a recess (48) for receiving the barb (39) of the corresponding hooking strip (37) of the first cover (30) when each of the buckling strips (45) is located beside the corresponding hooking strip (37) of the first cover (30) while the plate (31) of the first cover (30) extends substantially in a plane.

8. The safety syringe assembly according to claim 7, wherein each of the buckling strips (45) comprises an aperture (492) for receiving the barb (39) of the corresponding hooking strip (37) of the first cover (30) when each of the buckling strips (45) is engaged with the corresponding hooking strip (37) of the first cover (30) while the plate (31) of the first cover (30) is bent.

9. The safety syringe assembly according to claim 8, wherein each of the hooking strips (37) comprises a guiding edge (38), and each of the buckling strips (45) comprises a guiding edge (47), and the wings (12) are smoothly movable past the guiding edges (38/47) to facilitate the movement of the first and second covers (30/40) to the concealing position from the original position.

10. The safety syringe assembly according to claim 9, wherein the plate (41) of the second cover (40) comprises two slits (491) for receiving the guiding edges (38) of the hooking strips (37) when the buckling strips (45) are engaged with the hooking strips (37).

11. The safety syringe assembly according to claim 10, wherein each of the slits (491) of the plate (41) of the second cover (40) is in communication with the aperture (492) of a corresponding one of the buckling strips (45).

12. The safety syringe assembly according to claim 1, wherein each of the hooking strips (37) comprises a guiding edge (38), and each of the buckling strips (45) comprises a guiding edge (47), and the wings (12) are smoothly movable past the guiding edges (38/47) to facilitate the movement of the first and second covers (30/40) to the concealing position from the original position.

13. The safety syringe assembly according to claim 1, wherein the first cover (30) comprises two middle lateral strips (35) each connected to a corresponding one of the hooking strips (37), wherein each of the gaps (36) of the first cover (30) is made between a corresponding one of the middle lateral strips (35) and a corresponding one of the rear lateral strips (33).
